(19) United States
(12) Patent Application Publication
(10) Pub. No.: US 2023/0329371 A1
(43) Pub. Date: Oct. 19, 2023

(54) FACE SHIELD FOR USE WITH DENTAL LOUPES

(71) Applicant: KING SAUD UNIVERSITY, Riyadh (SA)

(72) Inventor: AMAL ABDULLAH IBRAHIM ALMOHAIMEDE, Riyadh (SA)

(21) Appl. No.: 17/019,332

(22) Filed: Sep. 13, 2020

(51) Int. Cl. A41D 13/11; A61B 1/24

(52) U.S. Cl. CPC A41D 13/1184; A61B 1/24; A61B 2090/502

(57) ABSTRACT

The face shield for use with dental loupes is a protective face shield for use by dentists, surgeons, medical practitioners and the like, and is adapted for usage when the user is wearing loupes. The face shield for use with dental loupes includes a curved transparent plate, with a substantially rectangular opening formed therethrough. A first pad is secured to an inner face of the curved transparent plate adjacent an upper end thereof, and is adapted for releasable positioning against the forehead of the user when the curved transparent plate is worn on the user’s head. The substantially rectangular opening is located adjacent the user’s eyes when the curved transparent plate is worn, such that the loupes worn by the user at least partially project therethrough. A first strap is connected to the laterally opposed side edges adjacent the first pad for holding the face shield on the user’s head.

### BACKGROUND

### 1. Field

The disclosure of the present patent application relates to medicine and dentistry, and particularly to a face shield adapted for use with loupes of the type used by dentists, surgeons and medical practitioners.

### 2. Description of the Related Art

A loupe is a simple, small magnification device used to see small details more closely. Unlike a magnifying glass, a loupe does not have an attached handle, and its focusing lens(es) are contained in an opaque cylinder or cone. Dental loupes aid dentists, hygienists, and dental therapists to devise accurate diagnoses of oral conditions and enhance surgical precision when completing treatment. Additionally, loupes can improve dentists’ posture, which can decrease occupational strain.

In use, dental caries (i.e., cavities), are most accurately identified by visual and tactile examination of a clean dry tooth. Magnification enables dentists to improve their ability to differentiate between a stain and a cavity. Cavities are rated and scored based on their visual presentation. If magnification is too high diagnosis becomes difficult due to the small field of view. Treatment of dental caries, periodontal disease, and pulpal disease are all aided by magnification. Additionally, the dental specialty of endodontics has performed the vast majority of research regarding magnification in dentistry. Because the identification of accessory canals in addition to the primary pulp canals is essential to complete nonsurgical root canal therapy, magnification provides dentists enhanced visualization to locate and treat more obscured canals. Such magnification is also useful in the treatment of periodontal disease, which is achieved by removing calculus deposits, plaque and therefore bacteria which causes inflammation and subsequently bone destruction. In severe cases, surgery to reduce pocket depth is indicated. Periodontists and hygienists must visualize plaque and calculus to remove it. Magnification can assist dentists and hygienists with identification and removal of plaque and calculus in addition to improving visualization for periodontal surgery.

As useful as dental loupes are, as indicated above, their usage with other equipment, particularly face shields, can be problematic. A typical dental loupe consists of additional magnifiers attached to eyeglasses or eyeglass-like frames, with the magnifiers projecting forward therefrom. With a typical face shield, there is little room between the wearer’s eyes (or eyeglasses) and the interior surface of the face shield. Thus, when the magnifiers of the dental loupe are added to the eyeglasses or eyeglass-like frames, they can strike the interior face of the face shield, causing misalignment of the magnifiers and creating uncomfortable pressure on the wearer’s face. Thus, a face shield for use with dental loupes solving the aforementioned problems is desired.

### SUMMARY

The face shield for use with dental loupes is a protective face shield for use by dentists, surgeons, medical practitioners and the like, and is adapted for usage when the user is wearing loupes. The face shield for use with dental loupes includes a curved transparent plate having an outer face, an inner face, opposed upper and lower ends, and laterally opposed side edges. A substantially rectangular opening is formed through the curved transparent plate and is laterally elongated. A first pad is secured to the inner face of the curved transparent plate adjacent the upper end thereof, and is adapted for releasable positioning against the forehead of the user when the curved transparent plate is worn on the user’s head. The substantially rectangular opening is located adjacent the user’s eyes when the curved transparent plate is worn, such that the loupes worn by the user at least partially project therethrough. A first strap is connected to the laterally opposed side edges adjacent the first pad, and is adapted for encircling the user’s head to keep the face shield for use with dental loupes in place on the user’s head.

Additionally, a second pad is secured to the inner face of the curved transparent plate. The second pad peripherally borders the substantially rectangular opening. A pair of laterally opposed tabs may be secured to the laterally opposed side edges of the curved transparent plate for connection to a second strap, which is also adapted to encircle the user’s head, providing additional support for the face shield for use with dental loupes.

These and other features of the present subject matter will become readily apparent upon further review of the following specification.

### BRIEF DESCRIPTION OF THE DRAWINGS

FIG. 1 is an environmental perspective view of a face shield for use with dental loupes.

FIG. 2 is an environmental front view of the face shield for use with dental loupes.

Similar reference characters denote corresponding features consistently throughout the attached drawings.

### DETAILED DESCRIPTION OF THE PREFERRED EMBODIMENTS

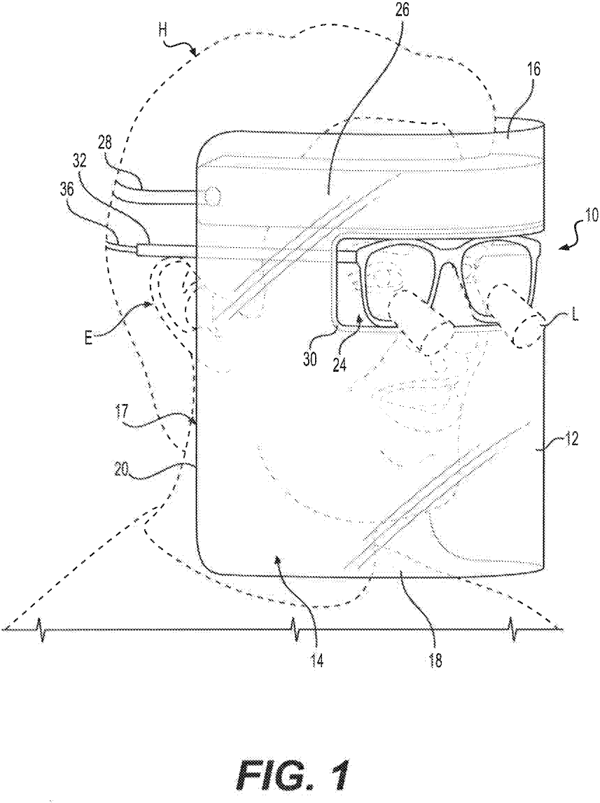

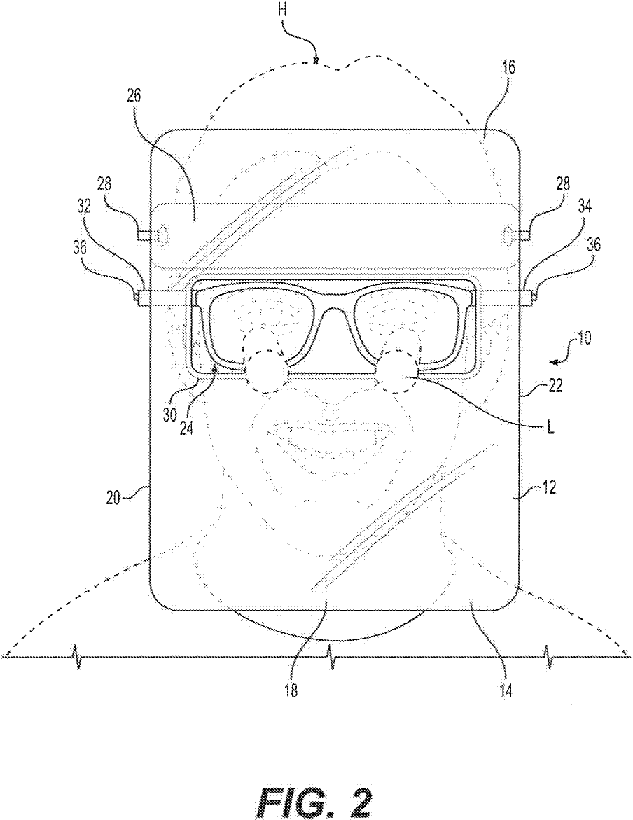

The face shield for use with dental loupes 10 is a protective face shield for use by dentists, surgeons, medical practitioners and the like, and is adapted for usage when the user is wearing loupes, such as exemplary loupes L of FIGS. 1 and 2. The face shield for use with dental loupes 10 includes a curved transparent plate 12 having an outer face 14, an inner face 17, opposed upper and lower ends 16, 18, respectively, and laterally opposed side edges 20, 22. It should be understood that loupes L are shown for exemplary purposes only. Further, it should be understood that the overall contouring and relative dimensions of curved transparent plate 12 are shown for exemplary purposes only, and that the curved transparent plate 12 may be manufactured in a variety of different sizes and shapes for use by a variety of different people and/or for a variety of different situations. In the non-limiting example of FIGS. 1 and 2, the curved transparent plate 12 extends from upper end 16 to lower end 18 such that, when worn, the curved transparent plate 12 extends from the user’s forehead to at least a portion of the user’s neck. In the non-limiting example of FIGS. 1 and 2, the curved transparent plate 12 extends laterally such that laterally opposed side edges 20, 22 cover approximately half of the user’s respective ears E. It should be further understood that the curved transparent face plate 12 may be formed from any suitable type of transparent protective material, such as transparent polycarbonate or the like.

A substantially rectangular opening 24 is formed through the curved transparent plate 12 and is laterally elongated. Although shown as a perfect rectangle in FIGS. 1 and 2, it should be understood that the substantially rectangular opening 24 may be varied in both shape and relative dimensions. For example, the corners of opening 24 could be curved. As another example, rectangular opening 24 could be defined by slightly curved borders, forming a slightly oval or elliptical shape. It should be understood that the overall contouring and relative dimensions of opening 24 are shown for exemplary purposes only, and that the opening 24 may be formed in a variety of different sizes and shapes for use by a variety of different people, for use with a variety of different types of loupes or other equipment, and/or for a variety of different situations.

A first pad 26 is secured to the inner face 17 of the curved transparent plate 12 adjacent the upper end 16. The first pad 26 is adapted for releasable positioning against the forehead of the user when the curved transparent plate 12 is worn on the user’s head H. The substantially rectangular opening 24 is located adjacent the user’s eyes when the curved transparent plate 12 is worn, such that the loupes L worn by the user at least partially project through substantially rectangular opening 24, as shown. A first strap 28 is connected to the laterally opposed side edges 20, 22 adjacent the first pad 26, and is adapted for encircling the user’s head H to keep the face shield for use with dental loupes 10 in place on the user’s head H. Although shown as rectangular in the non-limiting example of FIGS. 1 and 2, it should be understood that the first pad 26 may have any suitable size or shape, and the size and/or shape may be varied and/or customized to suit the particular user. Additionally, it should be understood that first pad 26 may be manufactured from any suitable material which is comfortable and non-irritating to the user’s skin, such as a light foam material or the like. Additionally, it should be understood that first strap 28 may be any suitable type of strap for holding the curved transparent plate 12 on the user’s head H, such as an adjustable and/or elastic strap or the like.

Additionally, a second pad 30 is secured to the inner face 17 of the curved transparent plate 12. The second pad 30 peripherally borders the substantially rectangular opening 24. As discussed above, it should be understood that the size and shape of the opening 24 may be varied, and that opening 24 is shown in FIGS. 1 and 2 for exemplary purposes only. Similarly, second pad 30 is shown for exemplary purposes only, and the shape and relative dimensions thereof may be varied such that second pad peripherally borders opening 24. Additionally, it should be understood that second pad 30 may be manufactured from any suitable material which is comfortable and non-irritating to the user’s skin, such as a light foam material or the like.

A pair of laterally opposed tabs 32, 34 may be secured to the laterally opposed side edges 20, 22 of the curved transparent plate 12, respectively, for connection to a second strap 36, which is also adapted to encircle the user’s head H, providing additional support for the face shield for use with dental loupes 10. It should be understood that second strap 36 may be any suitable type of strap for holding the curved transparent plate 12 on the user’s head 11, such as an adjustable and/or elastic strap or the like. Tabs 32, 34 are shown extending at approximately the level of the helix of the user’s ears E, although it should be understood that the positioning, shape and relative dimensions of tabs 32, 34 are shown for exemplary purposes only.

It is to be understood that the face shield for use with dental loupes is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

I claim:

1. A face shield for use with dental loupes, comprising:
a curved transparent plate having an outer face, an inner face, opposed upper and lower ends, and laterally opposed side edges, a substantially rectangular opening being formed through the curved transparent plate, the substantially rectangular opening being laterally elongated;
a first pad secured to the inner face of the curved transparent plate adjacent the upper end thereof, the first pad being adapted for releasable positioning against a forehead of a user when the curved transparent plate is worn on a head of the user, the substantially rectangular opening being located adjacent eyes of the user when the curved transparent plate is worn on the head of the user, such that dental loupes worn by the user at least partially project therethrough, wherein the first pad has a thickness and is sized such that an eyeglass frame of the dental loupes abuts the substantially rectangular opening when the dental loupe is also worn on the head of the user; and
a first strap connected to the laterally opposed side edges adjacent the first pad, the first strap being adapted to encircle the head of the user.

2. The face shield for use with dental loupes as recited in claim 1, further comprising a second pad secured to the inner face of curved transparent plate, the second pad peripherally bordering the substantially rectangular opening.

3. The face shield for use with dental loupes as recited in claim 1, further comprising:
a pair of laterally opposed tabs secured to the laterally opposed side edges of the curved transparent plate; and
a second strap connected to the laterally opposed tabs, the second strap adapted to encircle the head of the user.

4. A face shield for use with dental loupes, comprising:
a curved transparent plate having an outer face, an inner face, opposed upper and lower ends, and laterally opposed side edges, a substantially rectangular opening being formed through the curved transparent plate, the substantially rectangular opening being laterally elongated;
a first pad secured to the inner face of the curved transparent plate adjacent the upper end thereof, the first pad being adapted for releasable positioning against a forehead of a user when the curved transparent plate is worn on a head of the user, the substantially rectangular opening being located adjacent eyes of the user when the curved transparent plate is worn on the head of the user, such that dental loupes worn by the user at least partially project therethrough, wherein the first pad has a thickness and is sized such that an eyeglass frame of the dental loupes abuts the substantially rectangular opening when the dental loupe is also worn on the head of the user;
a second pad secured to the inner face of curved transparent plate, the second pad peripherally bordering the substantially rectangular opening; and
a first strap connected to the laterally opposed side edges adjacent the first pad, the first strap being adapted to encircle the head of the user.

5. The face shield for use with dental loupes as recited in claim 4, further comprising:
a pair of laterally opposed tabs secured to the laterally opposed side edges of the curved transparent plate; and
a second strap connected to the laterally opposed tabs, the second strap adapted to encircle the head of the user.

6. A face shield for use with dental loupes, comprising:
a curved transparent plate having an outer face, an inner face, opposed upper and lower ends, and laterally opposed side edges, a substantially rectangular opening being formed through the curved transparent plate, the substantially rectangular opening being laterally elongated;
a first pad secured to the inner face of the curved transparent plate adjacent the upper end thereof, the first pad being adapted for releasable positioning against a forehead of a user when the curved transparent plate is worn on a head of the user, the substantially rectangular opening being located adjacent eyes of the user when the curved transparent plate is worn on the head of the user, such that dental loupes worn by the user at least partially project therethrough, wherein the first pad has a thickness and is sized such that an eyeglass frame of the dental loupes abuts the substantially rectangular opening when the dental loupe is also worn on the head of the user;
a first strap connected to the laterally opposes side edges adjacent the first pad, the first strap being adapted to encircle the head of the user
a pair of laterally opposed tabs secured to the laterally opposed side edges of the curved transparent plate; and
a second strap connected to the laterally opposed tabs, the second strap adapted to encircle the head of the user.

7. The face shield for use with dental loupes as recited in claim 6, further comprising a second pad secured to the inner face of the curved transparent plate, the second pad peripherally bordering the substantially rectangular opening.
